(19) United States
(12) Patent Application Publication
(10) Pub. No.: US 2023/0003734 A1
(43) Pub. Date: Jan. 5, 2023

(54) IMMUNOMAGNETIC SEQUENTIAL ULTRAFILTRATION PLATFORM FOR ENRICHMENT AND PURIFICATION OF EXTRACELLULAR VESICLES FROM BIOFLUIDS

(71) Applicant: OHIO STATE INNOVATION FOUNDATION, Columbus, OH (US)

(72) Inventors: Eduardo Reategui, Columbus, OH (US); Andre Palmer, Columbus, OH (US); Jingjing Zhang, Columbus, OH (US); Richard Hickey, Columbus, OH (US); Thi Hien Luong Nguyen, Columbus, OH (US)

(21) Appl. No.: 17/778,765

(22) PCT Filed: Dec. 8, 2020
(86) PCT No.: PCT/US20/63695

§ 371 Date: May 20, 2022

Related U.S. Application Data
(60) Provisional application No. 62/956,375, filed on Jan. 2, 2020.

(51) Int. Cl. G01N 33/574; G01N 33/50; G01N 33/543

(52) U.S. Cl. CPC G01N 33/57488; G01N 33/5082; G01N 33/54326; C12Q 1/6886

(57) ABSTRACT

Methods for purifying and isolating extracellular vesicles (EVs) from a biofluid using a sequential processing. Tangential flow filtration is applied to the biofluid to increase the concentration of EVs in the biofluid. After this is achieved, enrichment mode is halted and the biofluid is processed in diafiltration mode to remove contaminants (up to 99.9%). After performing the tangential flow filtration step, the concentration of EVs in the biofluid is further increased by ultracentrifugal filtration. After performing the ultracentrifugal filtration step, EVs of a particular target type are separated from other EVs by immunomagnetic affinity separation. In some implementations, the methods are used to isolate and quantify tumor EVs for cancer evaluation. Additionally, these methods can be used with a scaling factor to quantify EVs from a less concentrated biofluid such as, for example, urine.

### RELATED APPLICATIONS

This application claims the benefit of U.S. Provisional Application No. 62/956,375, filed Jan. 2, 2020, and entitled “IMMUNOMAGNETIC SEQUENTIAL ULTRAFILTRATION PLATFORM FOR ENRICHMENT AND PURIFICATION OF EXTRACELLULAR VESICLES FROM BIOFLUIDS,” the entire contents of which are incorporated herein by reference.

### BACKGROUND

The present invention relates to systems and methods for isolating and purifying extracellular vesicles (EVs) from biofluids.

### SUMMARY

Extracellular vesicles (EVs) are submicron lipid particles endogenously shed from the surface of cells that have been identified in different biofluids. Analysis of EV molecular content has revealed a wide range of biological cargo, including proteins, mRNAs, microRNAs, and DNA fragments that resemble their cell of origin. Thus, for example, EVs derived from cancer cells have the potential to provide a much-needed source of non-invasive, molecular biomarkers for liquid biopsies. In some implementations, methods, and systems presented in this disclosure provide an approach referred to herein as “immunomagnetic sequential ultrafiltration” (iSUF) that includes a specific sequence of stages for purification and enrichment of EVs (specifically and nonspecifically). In iSUF, EVs present in different volumes of biofluids (e.g., 0.1 mL to 100 mL or greater) can be significantly enriched (e.g., 2 to 2000 times) with up to 99.9% removal of contaminating proteins (e.g., albumin, lipoproteins, globulins) and nucleic acids. In some implementations, the final stage of iSUF enables the separation of tumor-specific EVs by incorporating immunomagnetic beads specific to the target EVs. The iSUF methods and systems enable comprehensive downstream molecular analysis of isolated EVs unrestricted from the type of biofluids being analyzed.

In one embodiment, the invention provides a method for purifying and isolating extracellular vesicles from a biofluid using a sequential three-stage processing. Tangential flow filtration is applied to the biofluid in enrichment mode to increase the concentration of EVs in the biofluid. After this is achieved, enrichment mode is halted and the biofluid is processed in diafiltration mode to remove the majority of contaminants (up to 99.9%). After performing the tangential flow filtration step, the concentration of EVs in the biofluid is further increased by ultracentrifugal filtration. After performing the ultracentrifugal filtration step, EVs of a particular target type are separated from EVs of other types by immunomagnetic affinity separation.

In one embodiment, the invention provides a method of isolating a target type of extracellular vesicle from a biofluid. A concentration of extracellular vesicles in a biofluid is increased by applying tangential flow filtration to the biofluid. Then a buffer solution is added to the biofluid when the volume of the biofluid falls below a threshold. The tangential flow filtration continues on the biofluid after the buffer solution is added to the biofluid and run in diafiltration mode to remove unwanted contaminants, and then the biofluid is transferred to a centrifugal filtration unit after applying the tangential flow filtration to the biofluid with the added buffer solution. Centrifugal force is then applied to the biofluid in the centrifugal filtration unit to further increase the concentration of the extracellular vesicles in the biofluid. The biofluid is then transferred to an immunomagnetic affinity container with a plurality of magnetic beads, wherein antibodies affixed to the magnetic beads are configured to bind a target extracellular vesicle in the biofluid to the magnetic bead. The target extracellular vesicles are isolated from the biofluid by removing the magnetic beads from the biofluid and subsequently separating the target extracellular vesicles from the magnetic beads by elution.

In some embodiments, the method is used to evaluate a stage of cancer of a patient by isolating the tumor extracellular vesicles from the biofluid sample of the patient and then quantifying the tumor extracellular vesicles isolated from the biofluid sample. The stage of cancer is determined by identifying a stage of cancer corresponding to the quantification of the isolated tumor extracellular vesicles from the biofluid sample.

In another embodiment, the method is used to analyze extracellular vesicles extracted from different types of biofluids. A first sample of a first type of biofluid and a first sample of a second type of biofluid—both collected contemporaneously from the same patient—are provided and the EV isolation method is applied to both samples to isolate the target type of extracellular vesicles from each sample. A scaling factor is then calculated based on a quantification of the target type of extracellular vesicles isolated from the first sample of the first type of biofluid and a quantification of the target type of extracellular vesicles isolated from the first sample of the second type of biofluid. Subsequently, a second sample of the second type of biofluid is collected from the same patient at a second time and the method is again applied to isolate and quantify extracellular vesicles from the second sample of the second type of biofluid. An equivalent quantification of the target type of extracellular vesicles for the first type of biofluid is calculated by adjusting the quantification of the target type of extracellular vesicles isolated from the second sample of the second type of biofluid based on the calculated scaling factor. Accordingly, the method can be used to quantify conditions based on concentrations of extracellular vesicles while using a more readily available and less intrusively obtained biofluid such as, for example, urine instead of blood or serum.

Other aspects of the invention will become apparent by consideration of the detailed description and accompanying drawings.

### BRIEF DESCRIPTION OF THE DRAWINGS

FIG. 1 is a flowchart of a three-stage, sequential method for enrichment and isolation of specific extracellular vesicles (EVs) from biofluids according to one implementation.

FIG. 2 is a schematic diagram of a system for the tangential flow filtration stage of the method of FIG. 1.

FIG. 3A is a cross-sectional schematic diagram of a centrifugal filtration unit for the centrifugal filtration stage of the method of FIG. 1.

FIG. 3B is a schematic diagram of the centrifugal filtration performed using the unit of FIG. 3A.

FIG. 4A is a block diagram of a system for performing the immunomagnetic affinity selection stage of the method of FIG. 1.

FIG. 4B is a schematic diagram of a magnetic bead for immunomagnetic affinity selection bound to a plurality of the specific extracellular vesicles (EVs) in the method of FIG. 1.

FIG. 4C is a cross-sectional view of a plurality of magnetic beads bound to the specific target extracellular vesicles (EVs) in the immunomagnetic affinity selection stage of the method of FIG. 1.

FIG. 5 is a block diagram of an electrical control system for performing the method of FIG. 1 in some implementations.

FIG. 6 is a flowchart of a specific example of a method for performing the three-stage, sequential process of FIG. 1.

FIG. 7 is a flowchart of a method for evaluating and diagnosing cancer using the three-stage, sequential process of FIG. 1.

FIG. 8 is a flowchart of a method for determining a scaling for analysis of extracellular vesicles obtained from different types of biofluids using the three-stage, sequential process of FIG. 1.

FIG. 9 is a flowchart of an example of a method for therapeutic use of extracellular vesicles obtained by the three-stage, sequential process of FIG. 1.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Extracellular vesicles (EVs) are lipid particles present in nearly all biological fluids including blood, urine and cell culture media. The size of an EV can vary, for example, from 30 nm to a few microns. EVs are endogenously shed from the surface of cells through two distinct mechanisms, leading to different types of vesicles: multivesicular bodies (MVBs) and lipid vesicles. MVBs contain smaller vesicles that can fuse with the plasma membrane to release exosomes (e.g., 30 nm to 200 nm). Lipid vesicles bud directly from the plasma membrane as microvesicles (e.g., 200 nm to 1 μm). Large lipid bodies referred to as oncosomes (e.g., 1 μm to 5 μm) have also been classified as a type of EVs. EVs carry a wide variety of biological cargo, including proteins, RNA, and DNA fragments, giving EVs unique roles as delivery vehicles in regulating cell-to-cell communication. Tumor EVs, in particular, contain a select subset of proteins and nucleic acids that can precisely manipulate cellular microenvironments at distant sites to promote angiogenesis, invasiveness, immunosuppression, and metastasis.

EVs are more abundant than other circulating biomarkers and they are structurally more robust since the proteins and nucleic acids are contained inside the lipid vesicles. EVs can serve as diagnostic biomarkers and therapeutic carriers based on their various functions. In some implementations, tumor EV can be utilized as a biomarker to monitor cancer at different stages. In some implementations, EVs generated from non-invasive cells (e.g., red blood cells (RBCs), mesenchymal stem cells) possess advantageous features such as drug delivery vehicles that can cross tissue and cellular barriers, making them promising for drug delivery applications. However, tumor EVs present in biofluids are surrounded by massive amounts of normal EVs (secreted by healthy cells) and other biomolecules (e.g., albumin, lipoproteins, globulins) that influence diagnostic and therapeutic settings. Therefore, isolation and purification techniques for specific EVs (e.g., tumor EVs, RBCs-EVs, mesenchymal stem cell EVs) may be beneficial.

In some implementations, ultracentrifugation and density gradient methods may be used to process different biofluids. However, these techniques are labor-intensive, producing protein aggregate contaminants, and are nonspecific towards EV type. Moreover, in some cases, additional washing steps (e.g., further ultracentrifugation rounds) are required to purify EVs from contaminant proteins and aggregates. Other EV isolation methods (including, for example, polymeric or salt precipitation kits, size exclusion chromatography (SEC) columns (e.g., qEVs), and nano/microdevices) also have their limitations. For precipitation kits, the yield of EV recovery is low with a lack of specificity and poor purity. Although qEVs can remove protein contaminants from biofluids and can separate EVs into different size fractions with high purity, qEVs recover EVs at low yields and cannot distinguish tumor-specific EVs. The different sizes and molecular content of EVs create a challenge to purification and isolation techniques.

The systems and methods, such as described in the examples below, enable processing of small or large volumes of biofluids (e.g., 0.1 mL, 1 mL, 10 mL, 50 mL, 100 mL, greater than 100 mL). EVs are isolated specifically on-demand and with minimal protein and RNA contamination carryover (e.g., less than 0.1% albumin, lipoproteins, globulins). This high degree of purity in the isolated EVs enables, for example, the detection of small differences in target proteins and RNAs between study groups and individual patients.

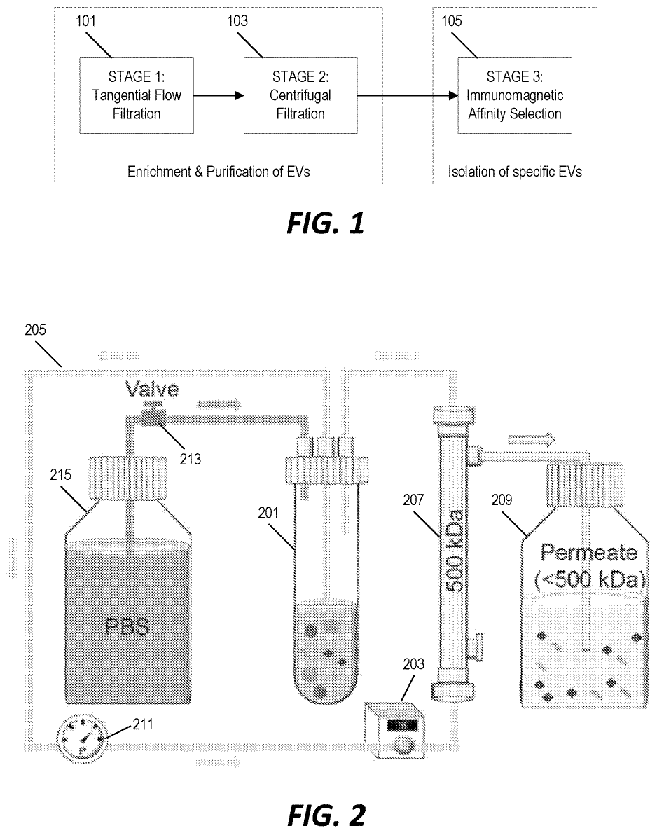

FIG. 1 illustrates a three-stage sequential process for isolation and purification of a specific target type of EV (e.g., tumor EVs). This process is referred to herein as immunomagnetic sequential ultrafiltration (iSUF). Stage 1 of the iSUF process includes a tangential flow filtration (TFF) (step 101) to remove free proteins, free nucleic acids, and other contaminants and to increase the concentration of EVs in the biofluid. Stage 2 includes applying centrifugal filtration (step 103) to the resulting biofluid sample volume after the TFF step to concentrate the sample further. Finally, Stage 3 uses immunomagnetic affinity selection (step 105) to isolate a particular target type of EV. Together, Stage 1 and Stage 2 provide enrichment and purification of EVs, but the resultant sample includes multiple different types of EVs. Accordingly, Stage 3 separates the specific target type of EVs from the other EVs.

FIG. 2 illustrates an example of a system for performing the Stage 1 tangential flow filtration of the iSUF process. The biofluid is transferred to a biofluid container 201. A peristaltic pump 203 circulates the biofluid sample from the biofluid container 201 through a tubing 205, through a hollow fiber filter 207, and back to the biofluid container 201 at a controlled flow rate. Relatively small particles pass through the membrane of the hollow fiber filter 207 and enter a permeate container 209. The larger particles remain in the biofluid sample and are returned to the biofluid container 201. The sample fractionation provided by the TFF stage depends on the membrane pore size (or molecular weight cut-off (MWCO)) of the hollow fiber filter 207, which should be large enough to allow free proteins and nucleic acids to pass through while small enough to retain EVs in the biofluid sample. A pressure sensor 211 monitors the fluid pressure on the tubing 205 and, in various implementations, may be used to control the operation of the peristaltic pump 203. A valve 213 is selectively opened to add a phosphate-buffered solution (PBS) from a PBS container 215 to the biofluid sample in the biofluid container 201. This facilitates diafiltration also known as buffer exchange.

In the system of FIG. 2, the Stage 1 TFF process includes two steps—an enrichment step and a diafiltration step. During the enrichment step, the concentration of EVs in the biofluid sample is increased and freely permeable molecules are partially removed through the hollow fiber filter 207. The diafiltration step further eliminates these molecules by controllably adding a washing buffer (e.g., PBS from the PBS container 215) to the biofluid sample by controllably opening the valve 213. The diafiltration step begins (i.e., by opening the valve 213) when the liquid level in the biofluid container 201 falls below a threshold. For example, the system may be configured to open the valve 213 to begin diafiltration when the liquid level in the biofluid container 201 is approximately equal to the sum of the dead volume of the TFF pump system (2 mL) and the liquid remaining in the tubing (5 mL). By adding the buffer to the biofluid sample when the volume reaches this threshold (7 mL), the EVs in the liquid are protected and continuous-mode operation of the TFF stage is ensured. Otherwise, EVs might not be stable when samples generate bubbles.

In some implementations, the enrichment step is performed before the diafiltration step because the contamination removal efficiency is proportional to the ratio of diafiltration buffer volume and the sample volume. For example, for larger samples of cell supernatant and urine, they are enriched down to the threshold volume of the TFF stage before the diafiltration step. In some implementations, the enrichment step is waived and the sample must be diluted to reach the threshold volume to avoid bubble formation before the diafiltration step. For example, a smaller serum sample is first diluted with PBS (e.g., a 0.1 to 0.5 mL sample is diluted to 7 mL) and then processed via diafiltration. In this manner, the performance of the TFF stage is made similar between different sample types.

The TFF stage process can be tuned for specific types of biofluids or target EVs, for example by adjusting variables such as flow rate, pressure, membrane pore size, membrane surface area, temperature, small or large biofluid volume, etc. In the example of FIG. 2, a 500 kDa filter is used as the hollow fiber filter 207. Filters with a smaller pore size (300 kDa) may also be used in some implementations because the majority of free proteins and nucleic acids in the cell culture supernatant, urine, and serum have molecular weights below 300 kDa. However, testing results have shown that both 300 and 500 kDa filters are able to remove up to 99.9% of free proteins and nucleic acids (with over 95% EV recovery) while the processing time of 300 kDa filters was 2-3 times higher than 500 kDa filters. Since processing time is critical for protecting EVs and their encapsulated molecular contents, the 500 kDa filter was preferred.

In some implementations, the biofluid processing by the TFF stage is performed at 4° C. to prevent EV degradation. Therefore, the shorter processing time and higher flow rate provided by the 500 kDa filter reduce the risk of degradation of the molecular content of EVs. However, as the pump (TFF stage) 203 flow rates are increased, there is a linear increase in shear rate being exerted on EVs which may damage their integrity. In one example, the TFF stage is operated with a maximum flow rate of 35 mL/min to maintain a shear rate along the filter 207 below 5000/sec to minimize shear-induced damaged to the EVs. The pressure was also controlled below 10 psi to protect the system and avoid leakage The relatively high flow rate (as compared to that which is achievable with a smaller pore-size filter), viscosity, and proteins in the fluid may generate a more substantial flow resistance that increased the pressure. The pressure resistance of the TFF stage illustrated in FIG. 2 was tested at a maximum of 35 mL/min flow rate with fetal bovine serum (FBS) at different concentrations, and the operation was stable if the protein concentration is below 15 mg/mL.

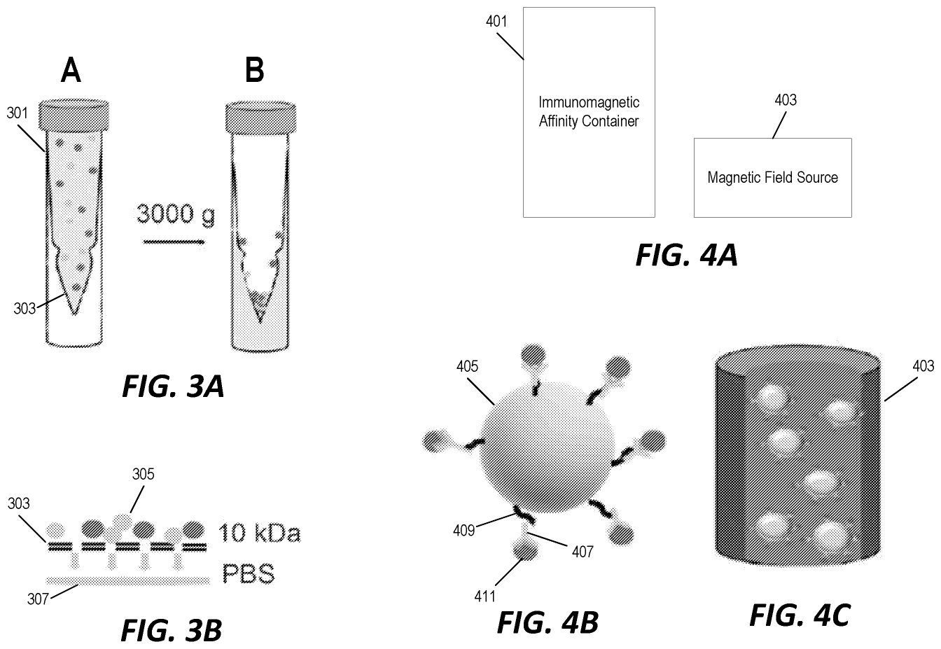

In Stage 2, ultracentrifugal filtration is used to concentrate the EVs in the sample further. FIG. 3A illustrates an example of an ultra-centrifugal filtration unit 301 with a membrane 303 positioned within the internal volume of the filtration unit 301. The sample is transferred to the filtration unit 301 on the upper side of the membrane 303 and the sample is filtered during centrifugation by the membrane 303. In the example of FIGS. 3A and 3B, ultra-centrifugal filtration units 301 include a membrane 303 with a 10 kDa MWCO. The ultra-centrifugal filtration units 301 are placed in a centrifuge that is then operated to apply a centrifugal force of 3000x gravity (g) to the sample. As centrifugation is applied, various different types of EVs 305 remain on the filter side of the membrane 303 while supernatant (e.g., PBS 307) passes through the membrane 303 and is removed from the biofluid sample. In this example, applying the centrifugation for 20 minutes to a 2 mL biofluid sample further concentrated the volume of the biofluid sample to 100 μL.

After Stage 1 and Stage 2, the resultant sample includes a high concentration of EVs of various types. In Stage 3, a particular target type of EVs (e.g., tumor EV) is isolated from the other EVs in the remaining sample volume using immunomagnetic affinity selection. As shown in FIG. 4A, the immunomagnetic affinity system includes an immunomagnetic affinity container 401 and a magnetic field source 403. The biofluid sample remaining after Stage 2 is transferred to the immunomagnetic affinity container 401 with a plurality of magnetic beads 405. FIG. 4B shows one example of a magnetic bead 405 with antibodies 407 coupled thereto by biotin 409. When these magnetic beads interact with the biofluid sample in the immunomagnetic affinity container 401, EVs of the specific target type are bound to the magnetic beads 405 by the antibodies 407. As shown in FIG. 4C, the magnetic beads 405 are attracted to the magnetic field source 403, and due to this attraction, the supernatant including all of the other types of EVs in the biofluid sample, can be removed from the biofluid sample. The EVs of the target type are then separated from the magnetic beads 405 by elution.

In the example of FIGS. 4A, 4B, and 4C, tumor EVs are selectively isolated from the 100 μL product from Stage 2 using 3 μm streptavidin-coated magnetic beads. EVs were able to be captured on beads functionalized with various biotinylated antibodies based on the biomarker expression level. EVs generated from glioblastoma (GBM), for which the epidermal growth factor receptor (EGFR) is highly expressed were used in this example. Before the isolation, the beads were functionalized with biotinylated EGFR antibody overnight at 4° C., followed by washing three times with PBS. The antibody was biotinylated using an EZ-Link micro Sulfo-NHS-biotinylation kit. Tumor EVs were then captured on the functionalized beads for 2 h at room temperature.

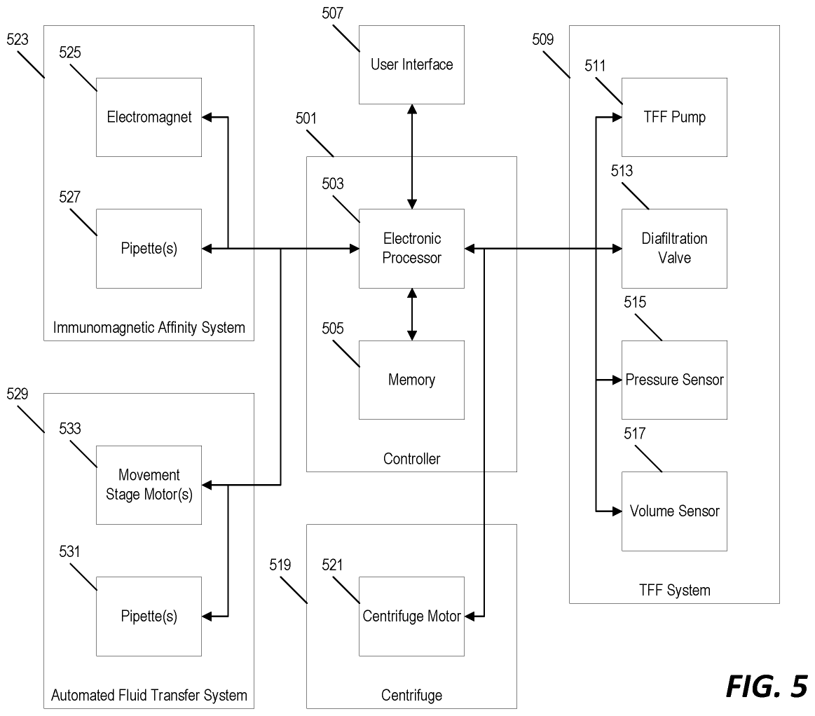

In some implementations, different stages of iSUF may be operated manually in sequence to perform the enrichment and purification of EVs as described above. However, in other implementations, the process may be wholly or partially automated by a computer-controlled system. FIG. 5 illustrates an example of a control system for an automated system for performing iSUF. A system controller 501 includes an electronic processor 503 and a non-transitory, computer-readable memory 505. The memory 505 stores instructions that are executed by the electronic processor 503 to provide functionality of the controller 501 (including, for example, the functionality described herein). The controller 501 is communicatively coupled to a user interface 507 configured to receive user inputs and/or display information to the user. The user interface 507 may include, for example, a display screen and a mouse or a touch-sensitive display screen.

In the example of FIG. 5, controller 501 is also communicatively coupled to the TFF stage 509 and configured to transmit control signals to various actuators and to receive sensor data from various sensors of the TFF stage 509. For example, the controller 501 may be configured to provide control signals to operate the TFF pump 511 and to selectively open/close the diafiltration valve 513. The controller 501 may also be configured to receive sensor data from a pressure sensor 515 indicative of a pumping pressure of the TFF stage and from a volume sensor 517 indicative of a volume of the liquid sample in the biofluid container 201. The controller 501 may similarly be communicatively coupled to a centrifuge unit 519 to provide control signals to operate a centrifuge motor 521. The controller 501 may also be communicatively coupled to an immunomagnetic affinity system 523 to selectively activate/deactivate an electromagnet 525 to apply a magnetic field to the immunomagnetic affinity container 401 and, in some implementations, to a robotic pipette system 527 to remove the supernatant while the magnetic field attracts the magnetic beads.

Some automated (or partially automated) systems may also include automated fluid transfer subsystem 529 to transfer the biofluid sample between the three system stages. For example, a robotic pipette system may include one or more pipette(s) 531 mounted to a robotic movement stage 533. To transfer the biofluid sample from the TFF biofluid container 201 to the centrifugal filtration unit 301, control signals are sent from the controller 501 (1) to the motors of the movement stage 533 to move the pipette to the TFF biofluid container 201, (2) to the pipette 531 to draw the biofluid sample from the TFF biofluid container 201, (3) to the motors of the movement stage 533 to move the pipette to one of the centrifugal filtration units 301, and (4) to the pipette 531 to dispense the biofluid sample into the centrifugal filtration unit 301. Similarly, to transfer the biofluid sample from the centrifugal filtration unit 301 to the immunomagnetic affinity container 401, control signals are sent from the controller 501 (1) to the motors of the movement stage 533 to move the pipette to the centrifugal filtration unit 301, (2) to the pipette 531 to draw the biofluid sample from the centrifugal filtration unit 301, (3) to the motors of the movement stage 533 to move the pipette to the immunomagnetic affinity container 401, and (4) to the pipette 531 to dispense the biofluid sample into the immunomagnetic affinity container 401.

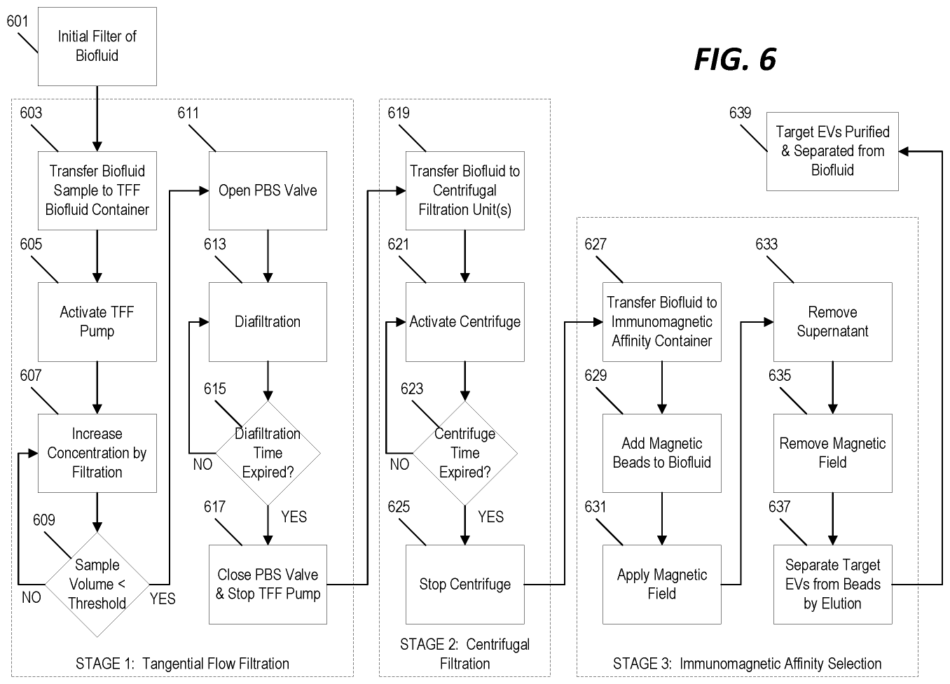

FIG. 6 illustrates in further detail an example of a method for performing iSUF using the systems described above. Before the first stage of the iSUF process begins, the biofluid sample is collected and pretreated by filtration (e.g., pore size of 1.0 μm) to remove aggregates (step 601). In some implementations, other pretreatment steps may be applied depending, for example, on the particular type of biofluids and/or the target EV type.

For example, in some implementations, cultured cells (e.g., U-251 glioblastoma (GBM) cells) may be incubated in RPMI medium (ThermoFisher Scientific, Waltham, Mass., USA) containing 10% FBS (fetal bovine serum) and 1% penicillin-streptomycin in a 37° C. and 5% CO₂ incubator. For a collection of EVs produced by U251 cells, U251 cells can be grown in T75 flasks (Corning, N.Y., USA) to obtain 90% cell confluence, followed by washing two times with PBS. The culture medium can then be replaced with EV-depleted medium (e.g., RPMI medium supplemented with 10% EV-depleted FBS and 1% penicillin-streptomycin) for 24 hours. In one particular example, EV-depleted FBS was the permeate of FBS filtered by a TFF (MWCO: 500 kDa) stage to remove bovine EVs originating from FBS. The collected supernatant was then centrifuged at 1000 RPM for 5 minutes to discard cell debris before further processing.

For serum, in some implementations, 8 to 10 mL of whole blood is collected into a BD SST Serum Tube. The tube is then immediately inverted 8-10 times and then gently placed upright for coagulation for at least 30 minutes (but no more than 2 hours). After that, the tubes are centrifuged at room temperature at 1100× gravity (g) for 10 minutes. Serum can then be aspirated carefully and stored in 1-mL aliquots at −80° C. (if not immediately processed by iSUF).

In some implementations, urine may be collected as the biofluid sample (e.g., either first-morning or second-morning urine) into sterilized 50-mL centrifuge tubes containing 4.2 mL protease inhibitor—a mixture of 1.67 mL 100 mM sodium azide (NaN₃), 2.5 mL PMSF, and 50 μL Leupeptin (Millipore Sigma). If iSUF processing is not performed immediately upon collection, the urine sample can be frozen (e.g., at −80° C.). However, in some implementations, frozen urine must be vortexed vigorously before iSUF processing.

After any appropriate pre-processing steps, Stage 1 of the iSUF processing begins by transferring the biofluid sample to the TFF biofluid container 201 (step 603). The TFF pump 203 is activated (step 605) and the TFF process increases the concentration of the biofluid sample by filtration (step 607). This continues until the sample volume falls below a defined threshold (step 609) and, in response, the PBS valve 213 is opened (step 611) adding the washing buffer to the biofluid sample. Diafiltration (613) continues until a defined time has elapsed (step 615) (or, in some other implementations, until the contaminants from the biofluid were completely removed). The PBS valve 213 is then closed. Finally, the biofluid sample is recovered in 2 mL and Stage 1 processing is completed (step 617).

After completion of Stage 1 processing, the resultant biofluid sample is transferred from the TFF biofluid container 201 to one or more centrifugal filtration units 301 (step 619). The centrifuge is then activated at a defined speed/force (step 621) and runs for a defined time. After the expiration of the defined centrifuge time period (step 623), the centrifuge is stopped and Stage 2 processing is completed. Again, as noted above, after completion of Stage 1 and Stage 2 processing in this specific sequence, the biofluid sample now includes a purified concentrated EVs with majority of proteins, nucleic acids, and other contaminants removed.

After completion of Stage 2 processing, the resultant biofluid sample is then transferred from the centrifugal filtration unit(s) 301 to the immunomagnetic affinity container 401 (step 627). Antibody-coated magnetic beads are added to the biofluid (step 629) and a magnetic field is selectively applied (step 631) to separate the target EVs (bound to the magnetic beads) from the rest of the biofluid sample. The other types of EVs are then removed from the biofluid sample as the supernatant of the biofluid sample (step 633). After the other types of EVs have been removed from the biofluid sample, the magnetic field is removed (step 635) and the target EVs are separated from the beads by elution (step 637).

The product of the iSUF process is a high concentration of EVs of the specific target type purified and separated from the other materials in the original biofluid (step 639). These isolated & purified EVs can then be used for various diagnostic, therapeutic, and research purposes. For example, as discussed above, tumor EVs in a biofluid can be used as a biomarker for diagnosing certain types of cancers. Similarly, a concentration of the tumor EVs in the biofluid can be used as a biomarker to indicate a particular stage or degree of cancer in a particular patient. Also, as discussed above, therapeutic EVs can be used as drug delivery vehicles for various types of diseases.

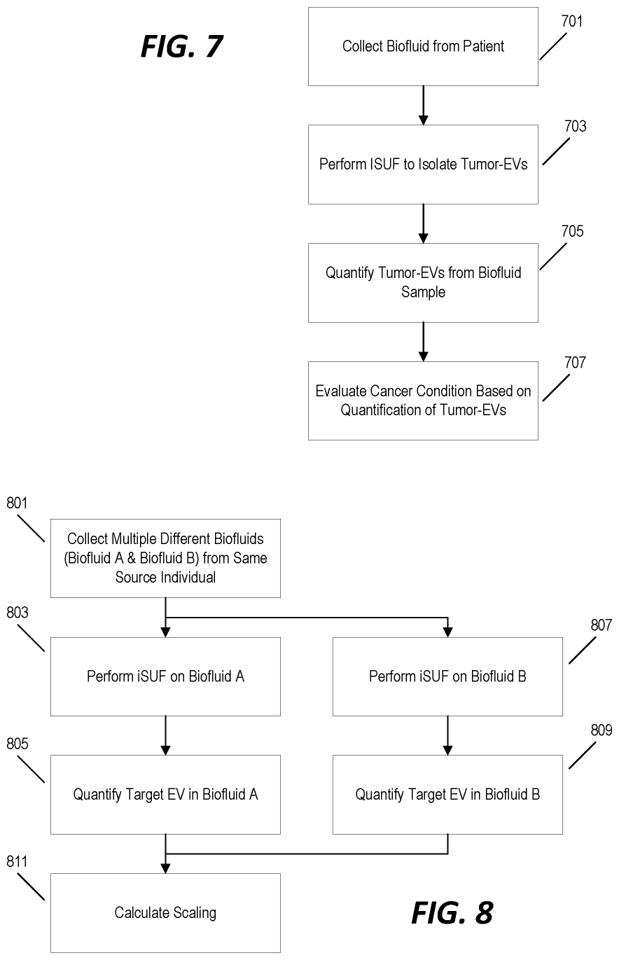

FIG. 7 illustrates one example of a method for using tumor EVs isolated and purified by the iSUF processing in order to diagnose cancer. First, a biofluid is collected from a patient (step 701) and iSUF is performed to isolate tumor EVs from the biofluid (step 703). The tumor EVs are then quantified (for example, by fluorescence processing) to determine a quantity of tumor EVs per volume in the original biofluid sample (step 705). This quantification of the tumor EVs can then be used as a metric to evaluate and/or characterize a cancer condition of the patient.

Different biofluids will exhibit different concentrations of particular EV types even within a single individual. Because the iSUF processing is able to purify and concentrate EVs effectively, biofluids that previously could not be used for EV analysis can now be used. For example, instead of using blood or serum for EV analysis, more diluted biofluids—such as, for example, urine—can be used for the same analysis. However, scaling may be necessary in order to determine an appropriate volume of the original biofluid for analysis and/or to compare quantified EV metrics obtained from different types of biofluids.

FIG. 8 illustrates a method for determining a scaling factor for comparing EV metrics from two different types of biofluids. First, multiple different biofluids are collected from the same source individual (e.g., the same patient) (step 801). The iSUF processing is applied to the first biofluid (i.e., Biofluid A) (step 803) and the target EVs in the first biofluid are quantified (step 805). The same iSUF processing is applied to the second biofluid (i.e., Biofluid B) (step 807) and the target EVs in the second biofluid are quantified (step 809). Because the biofluids are both collected from the same individual, there will be a correlation between the quantified EVs from the two different biofluids (for example, a proportional correlation). Accordingly, based on the quantification of the EVs from the two different biofluids, a scaling factor can be calculated to determine a volume of the first biofluid (i.e., Biofluid A) that would need to processed by iSUF to produce the same number of EVs as a particular volume of the second biofluid (i.e., Biofluid B) (step 811). By determining this scaling correlation, the less intrusive biofluid can be used for quantification of EVs for diagnosis and treatment methods.

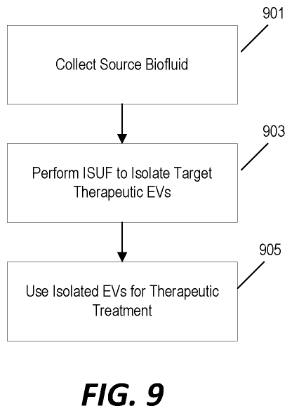

As noted above, isolated and purified EVs can also be used for therapeutic methods. For example, purified EVs such as RBC-EVs and MSC-EVs can be introduced in biofluids of a patient to transport a particular “cargo” and/or to cause a “reprogramming” of cells for therapeutic purposes. Engineered EVs can also be similarly used for therapeutic purposes. These therapeutic EVs can also be isolated by the iSUF processing to provide improved therapeutic efficacy. As illustrated in the example of FIG. 9, a biofluid containing the target therapeutic EV is collected (step 901) and iSUF is performed to isolate the target therapeutic EVs (step 903). These isolated EVs can then be used for therapeutic treatments (step 905). Although the examples above describe isolating target EVs from a “biofluid,” therapeutic EVs can be isolated by the iSUF process from actual biofluids or from laboratory-engineered fluids containing engineered/created therapeutic EVs.

Thus, the invention provides, among other things, a three-stage sequential processing method for purifying and isolating EVs of a particular target type from fluid samples. Other features and advantages of the invention are set forth in the accompanying drawings and the Appendix to this disclosure.

What is claimed is:

1. A method of isolating a target type of extracellular vesicle from a biofluid, the method comprising:
increasing a concentration of extracellular vesicles in a biofluid by applying tangential flow filtration to the biofluid;
adding a buffer solution to the biofluid when the volume of the biofluid falls below a threshold;
continuing to apply the tangential flow filtration to the biofluid after the buffer solution is added to the biofluid;
transferring the biofluid to a centrifugal filtration unit after applying the tangential flow filtration to the biofluid with the added buffer solution;
applying centrifugal force to the biofluid in the centrifugal filtration unit to further increase the concentration of the extracellular vesicles in the biofluid;
transferring the biofluid to an immunomagnetic affinity container with a plurality of magnetic beads, wherein antibodies affixed to the magnetic beads are configured to bind a target extracellular vesicle in the biofluid to the magnetic bead; and
isolating the target extracellular vesicle from the biofluid by removing the magnetic beads from the biofluid and subsequently separating the target extracellular vesicles from the magnetic beads by elution.

2. The method of claim 1, wherein the biofluid is blood.

3. The method of claim 1, wherein the biofluid is urine.

4. The method of claim 1, wherein the biofluid is serum.

5. The method of claim 1, wherein the biofluid is plasma.

6. The method of claim 1, wherein the biofluid is cerebrospinal fluid (CSF).

7. The method of claim 1, wherein increasing the concentration of extracellular vesicles in the biofluid by applying the tangential flow filtration to the biofluid includes applying the tangential flow filtration to a biofluid sample, and wherein the isolated target extracellular vesicles includes at least 95% of the extracellular vesicles from the biofluid sample.

8. A method of evaluating a stage of cancer of a patient by quantifying tumor extracellular vesicles, the method comprising:
isolating the tumor extracellular vesicles from a biofluid sample of the patient by applying the method of claim 1 to the biofluid sample;
quantifying the tumor extracellular vesicles isolated from the biofluid sample; and
determining the stage of cancer corresponding to the quantification of the isolated tumor extracellular vesicles from the biofluid sample.

9. A method of analyzing extracellular vesicles extracted from different types of biofluids, the method comprising:
providing a first sample of a first type of biofluid and a first sample of a second type of biofluid contemporaneously collected from a patient;
isolating a target type of extracellular vesicles from the first sample of the first type of biofluid by applying the method of claim 1 to the first sample of the first type of biofluid;
isolating the target type of extracellular vesicles from the first sample of the second type of biofluid by applying the method of claim 1 to the first sample of the second type of biofluid;
calculating a scaling factor based on a quantification of the target type of extracellular vesicles isolated from the first sample of the first type of biofluid and a quantification of the target type of extracellular vesicles isolated from the first sample of the second type of biofluid;
providing a second sample of the second type of biofluid collected from the patient at a second time subsequent to the collection of the first sample of the second type of biofluid;
isolating the target type of extracellular vesicles from the second sample of the second type of biofluid by applying the method of claim 1 to the second sample of the second type of biofluid; and
calculating an equivalent quantification of the target type of extracellular vesicles for the first type of biofluid by adjusting a quantification of the target type of extracellular vesicles isolated from the second sample of the second type of biofluid based on the calculated scaling factor.

10. The method of claim 9, wherein the second type of biofluid is urine.

11. The method of claim 10, wherein the first type of biofluid includes one selected from a group consisting of blood, serum, plasma, and cerebrospinal fluid (CSF).
